(19) United States
(12) Patent Application Publication
(10) Pub. No.: US 2006/0100492 A1
(43) Pub. Date: May 11, 2006

(54) Intra-esophageal catheter

(75) Inventors: Steven D. Hartle, Eden Prairie, MN (US); C. Thomas Bombeck, Lino Lakes, MN (US); Sundy M. Kinsley, Waconia, MN (US)

Correspondence Address: SHUMAKER & SIEFFERT, P. A., 8425 SEASONS PARKWAY, SUITE 105, ST. PAUL, MN, 55125, US

(73) Assignee: Medtronic, Inc., Minneapolis, MN (US)

(21) Appl. No.: 11/261,405

(22) Filed: Oct. 28, 2005

Related U.S. Application Data
(60) Provisional application No. 60/623,573, filed on Oct. 29, 2004.

(51) Int. Cl. A61B 5/05

(52) U.S. Cl. 600/350

(57) ABSTRACT

The disclosure is directed to an intra-esophageal catheter for sensing one or more physiological conditions within the esophagus, stomach or small bowel. The catheter includes features that facilitate catheter insertion and deployment, and reduce patient discomfort during deployment and indwelling use of the catheter. The intra-esophageal catheter has a main catheter section and a distal catheter section coupled to the main catheter body. The main catheter section is formed from a flexible material. The distal catheter section is formed from a material that is substantially less flexible than the flexible material of the main catheter section. The main catheter section and distal catheter section may be formed from different materials or different durometers of the same type of material.

This application claims the benefit of U.S. provisional application No. 60/623,573, filed Oct. 29, 2004, the entire content of which is incorporated herein by reference.

### TECHNICAL FIELD

The invention relates to medical devices and, more particularly, intra-esophageal catheters for sensing physiological conditions within the foregut, including the esophagus, stomach or small bowel.

### BACKGROUND

Gastroesophageal reflux occurs when stomach fluid, which typically includes stomach acids, intermittently flows from the stomach into the esophagus. It is common for most people to experience this fluid reflux occasionally as heartburn. Gastroesophageal reflux disease (GERD) is a clinical condition in which the reflux of stomach fluid into the esophagus is frequent enough and severe enough to impact a patient's normal functioning or cause damage to the esophagus.

In the lower part of the esophagus, where the esophagus meets the stomach, there is a muscular valve called the lower esophageal sphincter (LES). Normally, the LES relaxes to allow food to enter into the stomach from the esophagus. The LES then contracts to prevent stomach fluids from entering the esophagus. In GERD, the LES relaxes too frequently or at inappropriate times, allowing stomach fluids to reflux into the esophagus.

The most common symptom of GERD is heartburn. Acid reflux may also lead to esophageal inflammation, which causes symptoms such as painful swallowing and difficulty swallowing. Pulmonary symptoms such as coughing, wheezing, asthma, or inflammation of the vocal cords or throat may occur in some patients. More serious complications from GERD include esophageal ulcers and narrowing of the esophagus. The most serious complication from chronic GERD is a condition called Barrett's esophagus in which the epithelium of the esophagus is replaced with abnormal tissue. Barrett's esophagus is a risk factor for the development of cancer of the esophagus.

Accurate diagnosis of GERD is difficult but important. Accurate diagnosis allows identification of individuals at high risk for developing the complications associated with GERD. It is also important to be able to differentiate between gastroesophageal reflux, other gastrointestinal conditions, and various cardiac conditions. For example, the similarity between the symptoms of a heart attack and heartburn often lead to confusion about the cause of the symptoms.

Esophageal manometry, esophageal endoscopy, and esophageal pH monitoring are standard methods of measuring esophageal exposure to stomach acids and are currently used to diagnose GERD. A variety of endoscopic devices have been designed to monitor various parameters within the esophagus. One reliable technique for diagnosis of gastroesophageal reflux involves the deployment of a pH sensor to detect changes in intraesophageal pH in the lower esophagus over an extended monitoring period.

Existing techniques for intraesophageal pH sensing tend to rely on a pH sensing catheter or a telemetric pH sensing capsule that is implanted within the lower esophagus. An example of an implantable capsule is the Bravo™ pH monitoring system, marketed by Medtronic, Inc. of Minneapolis Minn. Examples of pH sensing catheters are the Z24™ and Slimline™ gastric and esophageal catheters marketed by Medtronic, Inc.

An intra-esophageal pH sensing catheter can support ambulatory esophageal pH monitoring when combined with an external recorder. An example of an external recorder is the Digitrapper™ data recorder marketed by Medtronic, Inc. The Digitrapper data recorder works in conjunction with a pH sensing catheter, and includes analysis and reporting software that facilitates comprehensive reporting of pH activity over a monitoring period.

A pH sensing catheter ordinarily is deployed trans-nasally into the esophagus to a point approximately 5 cm above the lower esophageal sphincter (LES). The proximal end of the catheter extends outside the patient's nose, and is usually taped down to the cheek and draped over the ear. Electrical conductors connect one or more sensors carried by the catheter to respective inputs on an external recorder carried by the patient. With such an arrangement, the patient is generally free to go about his daily routine.

Intra-esophageal catheters also can be used for diagnosis of conditions within the stomach and small bowel. In particular, a catheter may be deployed either trans-nasally or trans-orally to the esophagus, and then guided into the stomach or small bowel to sense physiological conditions. For example, such a catheter may be used to diagnose duodenogastric reflux.

U.S. Pat. No. 4,631,119 to Reichstein, U.S. Pat. No. 4,981,470 to Bombeck and U.S. Pat. No. 5,117,827 to Steube et al. disclose intra-esophageal catheters for sensing pH for diagnosis of gastroesophageal reflux. U.S. Pat. No. 6,689,056 to Kilcoyne et al. discloses an implantable probe for monitoring pH within the esophagus to diagnose gastroesophageal reflux.

### SUMMARY

In general, the invention is directed to an intra-esophageal catheter for sensing one or more physiological conditions within the foregut, such as the esophagus, stomach, or small bowel. The catheter includes a stiff distal section coupled to a relatively soft and flexible catheter body. The catheter includes one or more sensors configured to sense any of a variety of physiological conditions, such as pH, pressure, impedance, or temperature. The catheter also may include one or more sensors to sense physiological markers of gastroesophageal or duodenogastric reflux, such as bile, sodium, pepsin or pepsinogen.

Various embodiments of the present invention provide solutions to one or more problems existing in the prior art with respect to prior art catheter-based systems for sensing physiological conditions within the esophagus to diagnose GERD. These problems include the discomfort ordinarily associated with an indwelling nasoesophageal catheter. The presence of the catheter can be uncomfortable for the patient, and may cause irritation in the nose, throat and nasal passage. Also, patients may experience an increased swallowing urge when the catheter is in place, due to reflex stimulation. Increased swallowing can introduce excess air into the stomach, causing abdominal discomfort. In addition, in some cases, saliva intake associated with increased swallowing can result in increased pH within the esophagus.

Some of these problems may be compounded by the construction of the catheter. For example, a relatively stiff catheter may produce greater discomfort for the patient. Yet, as a further problem, if the catheter is too flexible, it is difficult for a care-giver to pass the catheter through the patient's nose, throat and esophagus. In this case, quick and precise deployment of the catheter may require significant time, training and experience.

Various embodiments of the present invention are capable of solving at least one of the foregoing problems. When embodied in a catheter for sensing one or more physiological conditions within the foregut, the invention includes features that facilitate catheter deployment and reduce patient discomfort during deployment and indwelling use of the catheter. In one embodiment, the invention provides a catheter having a main catheter section and a distal catheter section coupled to the main catheter section. The catheter may be useful not only within the esophagus, but also more generally within the foregut, including the esophagus, stomach and small bowel. For example, the catheter may be suitable for use within the esophagus to diagnose GERD, or for use within the stomach or small bowel to diagnose duodenogastric reflux.

The main catheter section is formed from a flexible material. The distal catheter section is formed from a material that is substantially less flexible than the flexible material of the main catheter section. The main catheter section and distal catheter section may be formed from different materials or different durometers of the same type of material. A sensor is positioned within the distal catheter section to sense one or more physiological conditions within an esophagus, stomach or small bowel of a patient. An electrical conductor is coupled to the sensor and extends from the sensor to a proximal end of the main catheter body. The invention also provides a method for forming an intra-esophageal catheter as described herein.

In one embodiment, the invention provides an intra-esophageal catheter comprising a main catheter section formed from a first material that is substantially flexible, a distal catheter section coupled to the main catheter section, the distal catheter section formed from a second material that is substantially less flexible than the first material, and a sensor within the distal catheter section to sense one or more physiological conditions within a foregut of a patient.

In another embodiment, the invention provides an intra-esophageal catheter comprising a substantially flexible main catheter section, a substantially stiff distal catheter section coupled to the main catheter section, and a sensor within the distal catheter section to sense one or more physiological conditions within a foregut of a patient.

In a further embodiment, the invention provides a method for forming an intra-esophageal catheter comprising coupling a substantially flexible main catheter section to a substantially stiff distal catheter section, the substantially stiff distal catheter section housing a sensor for sensing physiological conditions within a foregut of a patient.

In comparison to known foregut catheters, various embodiments of the invention may provide one or more advantages. For example, the stiff distal section of the disclosed catheter facilitates insertion and passage of the catheter within the patient's nose, throat and esophagus, as well as within the stomach and small bowel. The relative ease of catheter insertion and deployment reduces the amount of time needed for placement of the catheter, as well as patient discomfort associated with insertion. In addition, the amount of training and experience required by the care-giver can be reduced. Once the catheter is placed, the soft, flexible portion of the catheter reduces patient discomfort. Consequently, nose and throat irritation can be reduced. Furthermore, greater comfort may result in reduced swallowing frequency. In general, the invention provides a more convenient and comfortable intra-esophageal catheter for diagnosis of gastroesophageal reflux.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating a system for monitoring physiological conditions within the stomach or esophagus of a patient.

FIG. 2 is a schematic diagram of an intra-esophageal catheter for use in the system of FIG. 1.

FIG. 3 is a schematic diagram of the catheter of FIG. 2, illustrating compression of the catheter body.

FIG. 4 is an enlarged view of a distal catheter section of the catheter of FIGS. 2 and 3.

### DETAILED DESCRIPTION

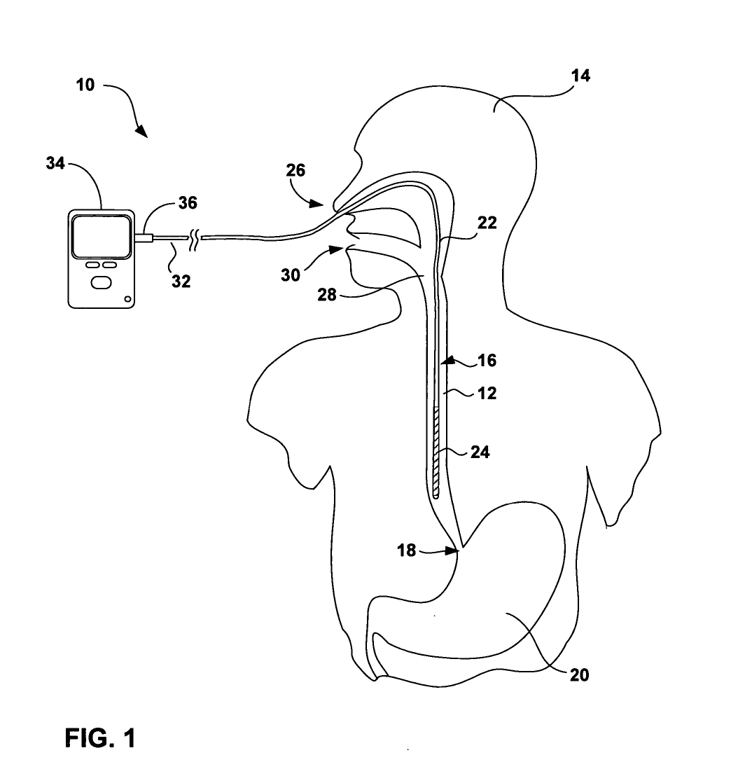

FIG. 1 is a schematic diagram illustrating a system 10 for monitoring physiological conditions within the esophagus 12 or stomach 20 of a patient 14. As shown in FIG. 1, system 10 includes an intra-esophageal catheter 16 for deployment of one or more sensors within esophagus 12 in the region of lower esophageal sphincter (LES) 18 above stomach 20. Alternatively, intra-esophageal catheter 16 may be used to deploy one or more sensors within stomach 20 or the small bowel. Therefore, although application of catheter 16 to diagnosis of conditions within esophagus 12, such as GERD, will be generally described herein, the catheter may be readily applicable to other locations and conditions, such as duodenogastric reflux, within the foregut of a patient 14.

Catheter 16 includes a main catheter section 22 and a distal catheter section 24 coupled to the main catheter section. In accordance with the invention, main catheter section 22 is formed from a flexible material, but distal catheter section 24 is formed from a material that is substantially less flexible than the flexible material forming the main catheter section. Hence, catheter 16 has a composite catheter construction with two distinct regions with different material properties.

Distal catheter section 24 is made relatively stiff to facilitate insertion and passage of catheter 16 through nasal passage 26, throat 28 and esophagus 12. Distal catheter section 24 may present an increased column strength when pushed through nasal passage 26, and provide better maneuverability. In particular, distal catheter section 24 may resist curling or bending as it is inserted. Unlike distal catheter section 24, main catheter section 22 is made relatively soft and flexible to promote patient comfort. The relative ease of insertion of the stiff distal catheter section 24 reduces the amount of time needed for placement of catheter 16, as well as patient discomfort associated with insertion. In addition, with stiff distal catheter section 24, a care-giver may require less experience and training to insert catheter 16.

Following placement of catheter 16, the soft, flexible main catheter section 22 reduces patient discomfort. Flexible main catheter section 22 is in contact with nasal passage 26, throat 28 and possibly a large extent of esophagus 12. Stiff distal catheter section 24 resides in the lower region of esophagus 12, or possibly into stomach 20 in other applications. With soft, flexible main catheter section 22, patient 14 may experience less irritation of nasal passage 26 and throat 28. For example, main catheter section 22 may better conform to surfaces within nasal passage 26 and throat 28, and exert less pressure on such surfaces. In addition, patient 14 may experience reduced swallowing frequency, which can reduce the amount of saliva ingested into esophagus 12.

As further shown in FIG. 1, catheter 16 may include a proximal end 32 coupled to an external recorder 34 via a connector 36. In particular, connector 36 electrically couples one or more electrical conductors from catheter 16 to corresponding inputs in external recorder 34. External recorder 34 receives continuous or periodic signals from a sensor or sensors carried by catheter 16, and records information based on the signals. The information may represent measurements of one or more physiological conditions within esophagus 12, or elsewhere in the foregut, such as the stomach. In some embodiments, external recorder 34 may be constructed in a manner similar to the Digitrapper™ data recorder marketed by Medtronic, Inc. External recorder 34 records the information for evaluation by a care-giver, e.g., for diagnosis of gastroesophageal reflux. For other foregut applications, external recorder 34 may aid in diagnosis of other disorders, such as duodenogastric reflux.

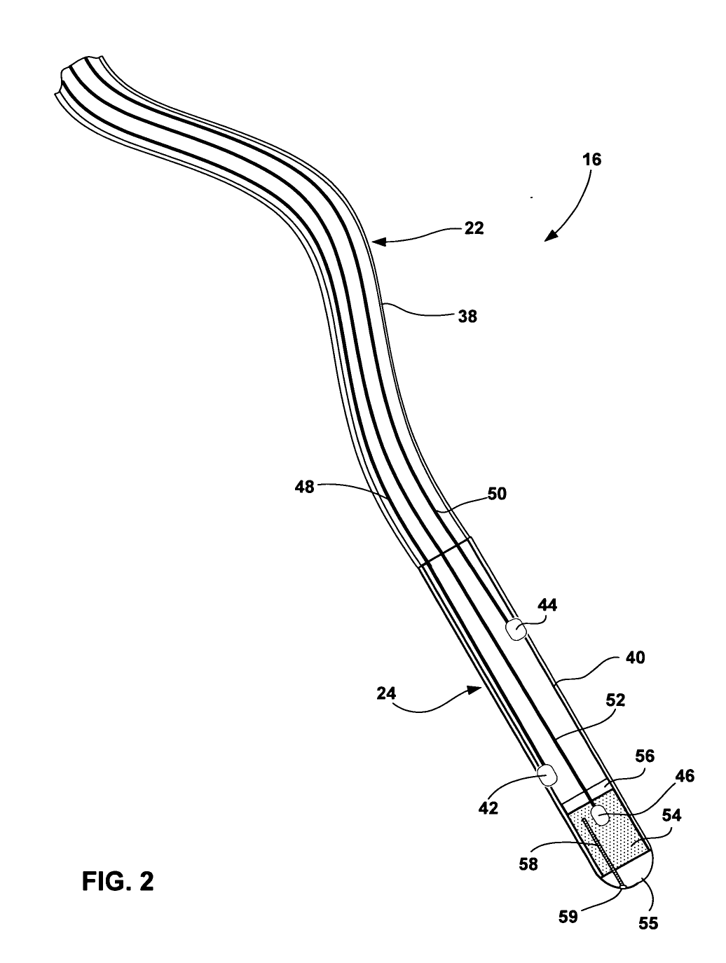

FIG. 2 is a perspective diagram of intra-esophageal catheter 16 for use in system 10 of FIG. 1. As shown in FIG. 2, main catheter section 22 is formed from a flexible material that permits the main catheter section to be easily bent. In this manner, main catheter section 22 can better conform to nasal passage 26 and throat 28 (FIG. 1) of patient 14. In addition, main catheter section 22 may be constructed to permit substantial deformation. In some embodiments, deployment of catheter 16 may be aided by an internal guidewire or steering mechanism. A guidewire may serve to add columnar strength to main catheter section 22 for insertion, and then be withdrawn once the catheter is in place. Accordingly, catheter 16 may include an inner lumen to accommodate a guidewire.

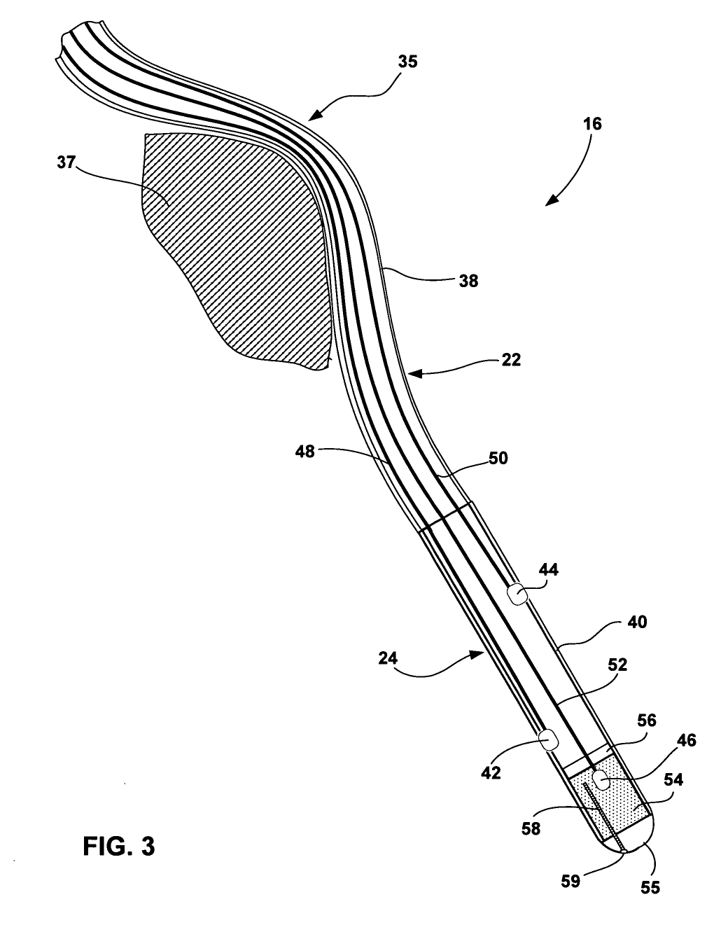

FIG. 3 is a schematic diagram of the catheter of FIG. 2, illustrating deformation of catheter 16. FIG. 3 is identical to FIG. 2, but illustrates a deformation point 35 at which an outer wall 38 of main catheter section 22 collapses. For example, outer wall 38 of catheter 16 may be compressed when it comes into contact with a surface 37 within nasal passage 26 or throat 28. Because catheter 16 typically will carry electrical conductors, rather than fluid carrying conduits, compression of main catheter section 22 of catheter 16 should not create a performance issue. On the contrary, general deformability of catheter 16 may be desirable for increased patient comfort, reducing pressure exerted on nasal passage 26 or throat 28 by the catheter. Distal catheter section 24 has an outer wall 40 constructed from a relatively stiff material, which may resist deformation and compression.

Main catheter section 22 and distal catheter section 24 may be constructed from different materials, or the same material with different hardness and flexibility characteristics. For example, main catheter section 22 and distal catheter section 24 may be constructed from a variety of polymeric materials such as polyvinyl chloride (PVC), silicone or polyurethane, or combinations of such materials. In some embodiments, blended combinations of materials may be formulated with different blend ratios to achieve different hardness characteristics appropriate for formation of main catheter section 22 or distal catheter section 24. Other possible materials for fabrication of main catheter section 22 or distal catheter section 24 may include polymers and polymer blends including, without limitation, nylon, polyether block amides (e.g., Pebax™), polyethylene terephthalate (PET), polyethylene, polypropylene, polyether etherketone (PEEK), polysulfone (PSU), polybutylene terephthalate (PBT), or polyphenylsulfone (PPSU).

Hence, main catheter section 22 and distal catheter section 24 may be formed from different materials, including different blended formulations of materials, or the same materials formulated to have different physical characteristics. In other words, the same type of material may be manufactured to exhibit different durometers. Accordingly, reference to a first material that forms the main catheter section 22 and a second material that forms distal catheter section 24 may refer to the use of different materials or the same type of material, provided that the materials used in sections 22 and 24 are different in terms of hardness characteristics.

In each case, main catheter section 22 and distal catheter section 24 preferably are fabricated as separate components and then coupled together to form catheter 16. Distal catheter section 24 may be bonded to main catheter section 22, for example, by a variety of techniques such as adhesive bonding, ultrasonic welding, or thermal crimping. Other suitable bonding techniques may include solvent bonding, thermal bonding, radio frequency (RF) bonding, insert (two-step) molding, extrusion, and mechanical bonding (e.g., by interference fit). Distal catheter section 24 and main catheter section 22 may be formed by conventional manufacturing processes such as extrusion, casting, or molding. The invention further contemplates a method for forming an intra-esophageal catheter 16, as described herein.

Outer wall 38 of main catheter section 22 and outer wall 40 of distal catheter section 24 may have substantially the same thickness, or the outer wall 38 of main catheter section 22 may be smaller. In this case, the thickness of outer wall 40 of distal catheter section 24 may be larger than the thickness of outer wall 38 of main catheter section 22 to thereby promote enhanced stiffness. Outer wall 38 of main catheter section 22 may have a durometer that is substantially less than the durometer of outer wall 40 of distal catheter section 24. As mentioned above, a variety of materials may be used to form main catheter section 22 and distal catheter section 24, such as polyvinyl chloride (PVC), silicone, and/or polyurethane. The soft, flexible main catheter section 22 may have a durometer between approximately 80 and 95 Shore A hardness, and more preferably approximately 90 Shore A hardness. The stiffer distal catheter section 24 may have a durometer of greater than or equal to approximately 90 Shore A hardness, and between approximately 40 to 70 Shore D. More preferably, distal catheter section 24 may have a durometer of greater than or equal to approximately 95 Shore A hardness, and between approximately 50 to 60 Shore D hardness.

Catheter 16 may have an overall length of approximately 130 to 190 cm. Distal catheter section 24 may have a length of approximately 8 to 35 cm, and more preferably 12 to 24 cm. In this case, main catheter section 22 has a length of approximately 100 to 162 cm, and more preferably 106 to 158 cm. Outer wall 38 and outer wall 40 each may have substantially the same thickness. For example, outer wall 38 and outer wall 40 each may have a thickness in a range of approximately 0.2 mm to 0.4 mm, and more preferably approximately 0.3 mm. Each section 22, 24 of catheter 16 may have substantially the same outer diameter, which may be approximately 1.1 to 2.3 mm, and more preferably 1.5 to 2.1 mm. Each section 22, 24 of catheter 16 also may have substantially the same inner diameter, which may be approximately 1.2 to 1.5 mm. In other embodiments, outer walls 38, 40 may have different thicknesses.

The outer surface of either section 22, 24 of catheter 16 may be coated with additional materials such as PTFE, silicone or other lubricating materials to facilitate insertion and promote patient comfort. In addition, the outer surface may be textured to include moisture pockets that trap liquid molecules on the surface of catheter 16 to enhance wetability and facilitate insertion.

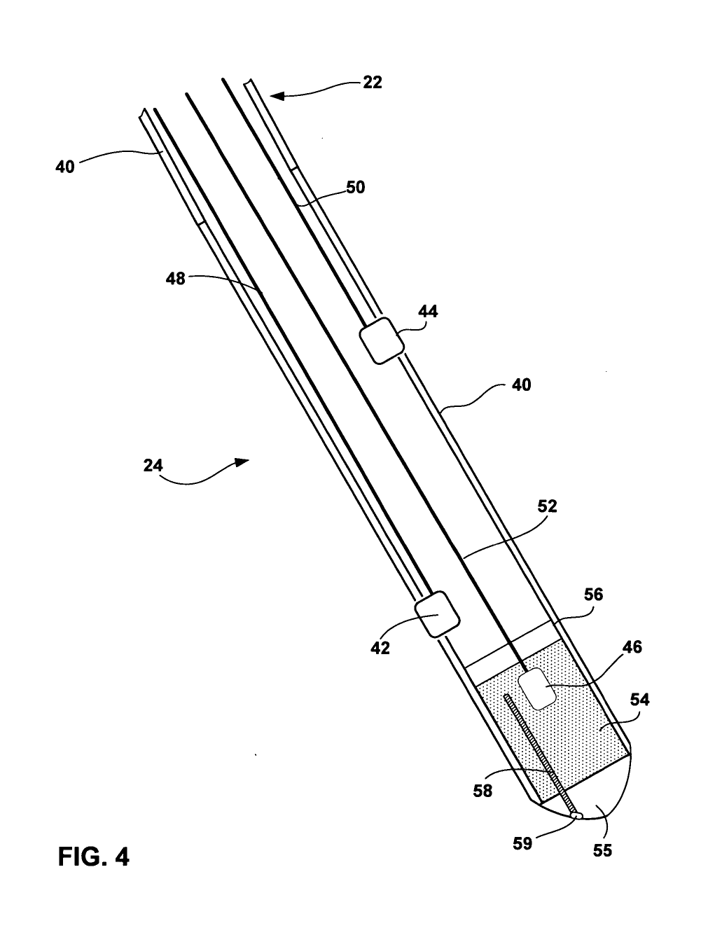

FIG. 4 is an enlarged view of a distal catheter section 24 of catheter 16 of FIGS. 2 and 3. As shown in FIGS. 2-4, catheter 16 includes one or more sensors within distal catheter section 24 to sense one or more physiological conditions within esophagus 12 or stomach 20. In the example of FIGS. 2-4, catheter 16 includes a first active pH sensor 42, a second active pH sensor 44 and an internal reference sensor 46, such as a reference electrode. Sensors 42, 44, 46 may correspond to any of a variety of conventional electrodes in the Z24 and Slimline pH monitoring catheters commercially available from Medtronic, Inc. One particular embodiment will be described herein for purposes of illustration.

Electrical conductors 48, 50, 52 are coupled to sensors 42, 44, 46, respectively, and extend from distal catheter section 24 to proximal end 32 of catheter 16. Electrical conductors 48, 50, 52 may be formed from insulated conductive wires suitable for medical grade applications, such as MP35N silver core wiring.

Sensors 42, 44, 46 are mounted within outer wall 40 of distal catheter section 24. The relatively stiff construction of outer wall 40 of distal catheter section 24 may protect sensors 42, 44, 46 from damage. Sensors 42, 44 are disposed within apertures in outer wall 40 and are exposed to the environment outside of catheter 16. In this manner, sensors 42, 44 are able to sense pH levels of contents within esophagus 12 or stomach 20. Sensors 42, 44 may be substantially identical, and may be formed as conventional antimony (Sb) sensors, such as die cast polycrystalline Sb. Dual sensors 42, 44 provide pH measurement at different heights within esophagus 12, which may be useful in diagnosis.

Reference sensor 46 is mounted within an end section 54 of catheter 16, between an end-cap seal 55 and an internal fluid seal 56. End section 54 is filled with an electrically conductive gel. Reference sensor 46 may take the form of a silver/silver chloride (Ag/AgCl) electrode. For example, AgCl may be formed on an Ag wire through electrochemical deposition techniques. A wicking medium 58 draws liquid from esophagus 12 into end section 54 through a porous plug 59 positioned within end-cap seal 55. In some embodiments, wall 40 of distal end section 24 may incorporate a porous plug in the end section 54, rather than a wicking medium 58. Water and ions move through the liquid junction created by the conductive gel. The conductive gel may be an electrolyte gel loaded with sodium chloride (NaCl) or another electrolyte. Reference sensor 46 may be an electrode that generates a stable electrical reference signal independent of the pH of the liquid drawn into end section 54 of the catheter.

Although an exemplary sensor embodiment has been described, the particular sensor implementation, and even the type of sensor, may vary. For example, other types of sensors useful in diagnosis of gastroesophageal reflux or duodenogastric reflux may be used in catheter 16. Examples include pressure sensors, temperature sensors, impedance-based sensors, optical sensors and flow sensors. Alternatively or in addition, a catheter as described herein may include one or more sensors to sense physiological markers of gastroesophageal or duodenogastric reflux, such as bile, sodium, pepsin or pepsinogen. In addition, in some embodiments, catheter 16 may include a combination of multiple sensors. In each case, regardless of the type of sensor, the relatively stiff distal catheter section 24 aids in insertion and deployment of the catheter to a desired position within the foregut, i.e., the esophagus, stomach or small bowel, while the more flexible main catheter section 22 promotes patient comfort and tolerance over the course of a monitoring period.

Various embodiments of the invention have been described. Various modifications may be made without departing from the scope of the claims. These and other embodiments are within the scope of the following claims.

1. An intra-esophageal catheter comprising:
a main catheter section formed from a first material that is substantially flexible;
a distal catheter section coupled to the main catheter section, the distal catheter section formed from a second material that is substantially less flexible than the first material; and
a sensor within the distal catheter section to sense one or more physiological conditions within a foregut of a patient.

2. The catheter of claim 1, further comprising an electrical conductor coupled to the sensor and extending from the sensor to a proximal end of the main catheter section.

3. The catheter of claim 1, wherein the first and second materials are the same type of material, and wherein the first and second materials have different durometers.

4. The catheter of claim 1, wherein the first and second materials are different types of materials.

5. The catheter of claim 1, wherein the main catheter section has a hardness in a range of approximately 80 Shore A to approximately 95 Shore A, and the distal catheter section has a hardness greater than a hardness of the main catheter section, and wherein the hardness of the distal catheter section is greater than or equal to approximately 90 Shore A.

6. The catheter of claim 1, wherein the main catheter section has a hardness of approximately 90 Shore A, and the distal catheter section has a hardness greater than a hardness of the main catheter section, and wherein the hardness of the distal catheter section is approximately 50 to approximately 60 Shore D.

7. The catheter of claim 1, wherein the first and second materials include at least one of polyvinylchloride, polyurethane, or silicone.

8. The catheter of claim 1, wherein walls defined by the main catheter section and the distal catheter section have substantially equal thicknesses.

9. The catheter of claim 1, wherein walls defined by the main catheter section and the distal catheter section have different thicknesses.

10. The catheter of claim 1, wherein the main catheter section and the distal catheter section have a combined length of approximately 130 to approximately 190 cm, and the distal catheter section has a length of approximately 12 to approximately 24 cm, and wherein each of the main catheter section and the distal catheter section has an outer diameter of approximately 1.1 to approximately 2.3 mm.

11. The catheter of claim 1, wherein the sensor includes a pH electrode, the catheter further comprising a reference electrode within the distal catheter section.

12. The catheter of claim 1, wherein the sensor includes an electrical impedance sensor.

13. The catheter of claim 1, wherein the sensor includes two or more sensors.

14. An intra-esophageal catheter comprising:
a substantially flexible main catheter section;
a substantially stiff distal catheter section coupled to the main catheter section; and
a sensor within the distal catheter section to sense one or more physiological conditions within a foregut of a patient.

15. The catheter of claim 14, further comprising an electrical conductor coupled to the sensor and extending from the sensor to a proximal end of the main catheter section.

16. The catheter of claim 14, wherein the main catheter section and the distal catheter section are formed from the same type of material, and wherein the main catheter section and the distal catheter section have different durometers.

17. The catheter of claim 14, wherein the main catheter section and the distal catheter section are formed from different types of materials.

18. The catheter of claim 14, wherein the main catheter section has a hardness in a range of approximately 80 Shore A to approximately 95 Shore A, and the distal catheter section has a hardness greater than a hardness of the main catheter section, and wherein the hardness of the distal catheter section is greater than or equal to approximately 90 Shore A.

19. The catheter of claim 14, wherein the main catheter section has a hardness of approximately 90 Shore A, and the distal catheter section has a hardness greater than a hardness of the main catheter section, and wherein the hardness of the distal catheter section is approximately 50 to approximately 60 Shore D.

20. The catheter of claim 14, wherein walls defined by the main catheter section and the distal catheter section have substantially equal thicknesses.

21. The catheter of claim 14, wherein walls defined by the main catheter section and the distal catheter section have different thicknesses.

22. The catheter of claim 14, wherein each of the main catheter section and the distal catheter section include at least one of polyvinylchloride, polyurethane, or silicone.

23. The catheter of claim 14, wherein the main catheter section and the distal catheter section have a combined length of approximately 130 to approximately 190 cm, and the distal catheter section has a length of approximately 12 to approximately 24 cm, and wherein each of the main catheter section and the distal catheter section has an outer diameter of approximately 1.1 to approximately 2.3 mm.

24. The catheter of claim 14, wherein the sensor includes a pH electrode, further comprising a reference electrode within the distal catheter section.

25. The catheter of claim 14, wherein the sensor includes an electrical impedance sensor.

26. The catheter of claim 14, wherein the sensor includes two or more sensors.

27. A method for forming an intra-esophageal catheter comprising coupling a substantially flexible main catheter section to a substantially stiff distal catheter section, the substantially stiff distal catheter section housing a sensor for sensing physiological conditions within a foregut of a patient.

28. The method of claim 27, wherein coupling includes one of adhesively bonding the main catheter section to the distal catheter section, or ultrasonically welding the main catheter section to the distal catheter section.

29. The method of claim 27, wherein the main catheter section and the distal catheter section are formed from the same type of material, and wherein the main catheter section and the distal catheter section have different durometers.

30. The method of claim 27, wherein the main catheter section and the distal catheter section are formed from different types of materials.

31. The method of claim 27, wherein the main catheter section has a hardness in a range of approximately 80 Shore A to approximately 95 Shore A, and the distal catheter section has a hardness greater than a hardness of the main catheter section, and wherein the hardness of the distal catheter section is greater than or equal to approximately 90 Shore A.

32. The method of claim 27, wherein the main catheter section has a hardness of approximately 90 Shore A, and the distal catheter section has a hardness greater than a hardness of the main catheter section, and wherein the hardness of the distal catheter section is approximately 50 to approximately 60 Shore D.

33. The method of claim 27, wherein walls defined by the main catheter section and the distal catheter section have substantially equal thicknesses.

34. The method of claim 27, wherein walls defined by the main catheter section and the distal catheter section have different thicknesses.

35. The method of claim 27, wherein each of the main catheter section and the distal catheter section includes at least one of polyvinylchloride, polyurethane, or silicone.

36. The method of claim 27, wherein the main catheter section and the distal catheter section have a combined length of approximately 130 to approximately 190 cm, and the distal catheter section has a length of approximately 12 to approximately 24 cm, and wherein each of the main catheter section and the distal catheter section has an outer diameter of approximately 1.1 to approximately 2.3 mm.

37. The method of claim 27, wherein the sensor includes a pH electrode, and the catheter further comprises incorporating a reference electrode within the distal catheter section.

38. The method of claim 27, wherein the sensor includes two or more sensors.
